(19) United States
(12) Patent Application Publication
(10) Pub. No.: US 2026/0263800 A1
(43) Pub. Date: Sep. 10, 2026

(54) DRY EYE TREATMENT DEVICE

(71) Applicant: LUMENIS BE LTD., YOKNEAM (IL)

(72) Inventors: May Kleinman Ben Tsvi; Yair Mandelstam-Manor

(21) Appl. No.: 19/166,103

(22) PCT Filed: Mar. 21, 2024
(86) PCT No.: PCT/IB2024/052733

§ 371 Date: Sep. 17, 2025

Related U.S. Application Data
(60) Provisional application No. 63/454,332, filed on Mar. 24, 2023.

(51) Int. Cl. A61N 1/36; A61N 1/06; A61N 1/40

(52) U.S. Cl. CPC A61N 1/36046; A61N 1/06; A61N 1/40

(57) ABSTRACT

A device for treating an eyelid with radio frequency (RF) radiation, comprising posterior component configured to be positioned over the internal side of the eyelid and an anterior component configured to be positioned on the external side of the eyelid. The device further comprising at least two electrodes, comprising any combination of one or more posterior electrodes mounted on said posterior component to be positioned on said internal side of the eyelid and/or one or more anterior electrodes mounted on said anterior component to be positioned on said external side of the eyelid, each said electrode configured to be positioned over an underlying zone of the eyelid to provide an RF signal between at least two electrodes.

### FIELD OF THE INVENTION

The present invention is in the medical field and relates to devices utilizing radio-frequency (RF) for therapy, and in particular relates to an RF therapy device for treatment of the eye.

### BACKGROUND TO THE INVENTION

Dry eye disease is a common condition resulting in uncomfortable feeling and may also lead to inflammation and damage of the eye's ocular surface. It occurs when the glands responsible for producing secretions become dysfunctional and/or when the secretions are not enough in quantity or quality, therefore not being able to provide adequate lubrication and protection to the ocular surface.

Available treatments for dry eye include eye drops and lifestyle changes such as controlling the time spent in front of screens. Other recent treatments include treatment by light energy, such as described in WO19186571 A1, or electromagnetic energy and specifically RF energy, such as described in WO2017072575 and U.S. 2017333249.

### SUMMARY

In accordance with a first aspect of the presently disclosed subject matter, there is provided a device for treating an eyelid with RF radiation, the device comprising:
a posterior component configured to be positioned over the internal side of the eyelid and an anterior component configured to be positioned on the external side of the eyelid;
at least two electrodes, comprising any combination of one or more posterior electrodes mounted on said posterior component to be positioned on said internal side of the eyelid and/or one or more anterior electrodes mounted on said anterior component to be positioned on said external side of the eyelid, each said electrode configured to be positioned over an underlying zone of the eyelid;
the device being configured for the at least two electrodes to provide an RF field to the eyelid along one or more of a coronal, transverse, and sagittal axis of the eyelid when an RF signal is applied between said at least two electrodes.

In some embodiments, the device further comprises a clamping mechanism configured to move the posterior and anterior components away from each other, thereby enabling insertion and removal of the device from the eyelid, and to compress the posterior and anterior components against each other, thereby securing said at least two electrodes to the respective underlying zones. In some embodiments, the clamping mechanism is manually operated. In some embodiments, the clamping mechanism is electrically operated. In some embodiments, the clamping mechanism is magnetically operated.

In some embodiments, when the clamping mechanism is activated, the posterior and anterior components are moved away from each other, and when the clamping mechanism is deactivated, the posterior and anterior components are compressed against each other.

In some embodiments, the at least two electrodes comprise two anterior electrodes positioned on said external side of the eyelid, the device thereby being configured to provide said RF field along the transverse axis of the eyelid. The at least two electrodes may comprise at least three anterior electrodes positioned on said external side of the eyelid along the transverse axis with a fixed distance between each two adjacent anterior electrodes, the device thereby being configured to provide one of the following effects:
a) a sliding RF field along the transverse axis of the eyelid by sequentially activating two adjacent anterior electrodes of the at least three anterior electrodes;
b) a length-varying RF field along the transverse axis of the eyelid by activating at each given time the most right electrode or the most left electrode of the anterior electrodes at a first polarity while sequentially activating the rest of the anterior electrodes at an opposite second polarity.

In some embodiments, the at least two electrodes comprise at least one anterior electrode positioned on said external side of the eyelid and at least one posterior electrode positioned on said internal side of the eyelid; the device thereby being configured to provide said RF field along the sagittal axis of the eyelid between the at least one anterior and at least one posterior electrodes.

In some embodiments, at least two electrodes comprise two anterior electrodes, the device thereby being configured to alternate said RF field along the sagittal axis of the eyelid between each of said two anterior electrodes and said at least one posterior electrode.

In some embodiments, the device is disposable. In some embodiments, the device is at least partially disposable.

In some embodiments, the device comprises a disposable sleeve configured to cover at least said at least two electrodes during treatment.

In an aspect of the invention, there is a device wherein the posterior component is electrically inactive, and the anterior component comprises two electrodes that have opposite polarities to each other, such that when an RF signal is applied between the two electrodes, an RF field propagates between the two electrodes. Also, there is a device wherein: the posterior component comprises a posterior electrode with a first polarity, and the anterior component comprises two anterior electrodes both comprising a second polarity; and the posterior electrode and the two anterior electrodes are further configured to apply the RF signal between the posterior electrode alternately to each of the two anterior electrodes.

In another aspect of the invention there is a device wherein the posterior electrode is further configured to cyclically switch from a first polarity to a second polarity and the two anterior electrodes are further configured to cyclically switch from a second polarity to a first polarity in sync with the switch of the posterior electrode. Also a device, wherein the posterior component is electrically inactive, and the anterior component comprises one anterior electrode fixed in a first polarity, and at least two additional anterior electrodes and the at least two additional anterior electrodes are configured to switch between a second plurality and an electrically inactive status.

In a final aspect, there is a device for treating an eyelid, comprising: a posterior component configured to be positioned over the internal side of the eyelid and an anterior component configured to be positioned on the external side of the eyelid; said posterior component to be positioned on said internal side of the eyelid said anterior component to be positioned on said external side of the eyelid; at least one of the posterior component or the anterior component configured to be a heating element to provide heating of the Meibomian glands of the eyelid. The device, wherein the device is further configured to allow a user to compress an eyelid between the posterior component and the anterior component. Also the device further comprising at least two electrodes, comprising any combination of one or more posterior electrodes mounted on said posterior component and/or one or more anterior electrodes mounted on said anterior component, each said electrode configured to be positioned over an underlying zone of the eyelid.

### BRIEF DESCRIPTION OF THE DRAWINGS

FIG. 1 depicts a Cartesian coordinate system labeled according to terminology used in the present invention;

FIGS. 2A-B depict an eyelid treatment device, according to some non-limiting embodiments of the present invention;

FIGS. 3A-B depict an electromagnetically controlled RF eyelid treatment device, according to some non-limiting embodiments of the present invention;

FIG. 4 depicts a bipolar RF eyelid treatment device, according to some non-limiting embodiments of the present invention.

FIG. 5 depicts a tripolar sagittal RF eyelid treatment device, according to some non-limiting embodiments of the present invention;

FIG. 6 depicts a tripolar transverse-sagittal RF eyelid treatment device, according to some non-limiting embodiments of the present invention;

FIG. 7 depicts a dynamic field-length bipolar RF eyelid treatment device, according to some non-limiting embodiments of the present invention;

FIG. 8 depicts a dynamic field-position bipolar RF eyelid treatment device, according to some non-limiting embodiments of the present invention; and

FIG. 9 depicts non-limiting examples of RF directions discussed in the present invention.

### DETAILED DESCRIPTION

The present disclosure is related to a treatment probe using RF energy to heat the Meibomian glands that have an essential role in adequate meibum production, to treat Meibomian gland dysfunction (MGD) and facilitate expression of the meibomian glands, to relief dry eye signs and symptoms.

The treatment probe may include a stainless steel or other suitable metal tip to be positioned on the eyelid.

The treatment probe, fully or partially, can be made disposable or for multi-use. Additionally, or alternatively, a disposable sleeve can be used to temporarily cover/envelop at least the parts of the probe that come into contact with the body. This alleviates the need for repetitive sterilization of those parts.

The term “region,” as used in this disclosure, refers to a surface area or a volume of the eyelid under treatment with an RF eyelid treatment device of the present invention. The region may be located between electrodes of the device.

The term “Underlying zone” refers to a region of the eyelid over which an electrode of the device is positioned. The electrode may or may not be in electrical contact with the underlying zone.

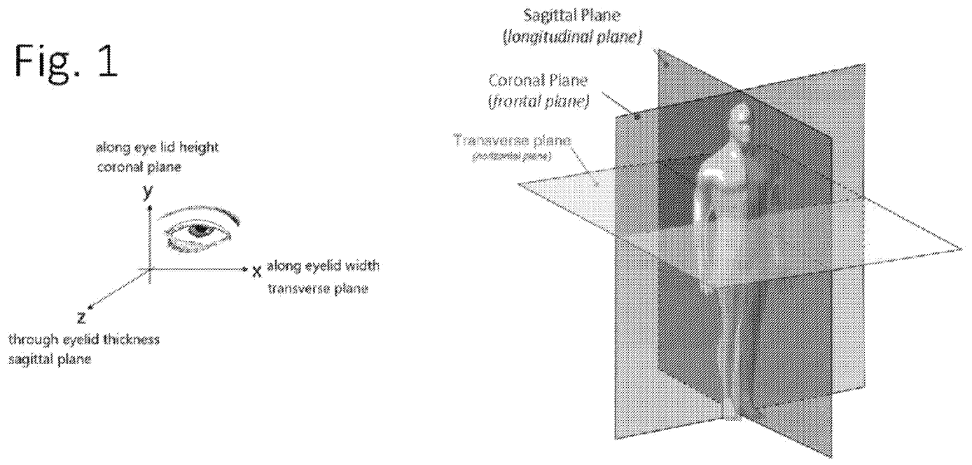

Reference is now made to FIG. 1, depicting a Cartesian coordinate system labeled according to terminology used in this disclosure. The x-axis is along the width of the eyelid, along the width of the transverse plane (also called the horizontal plane). The y-axis is along the height of the eyelid, along the height of the coronal plane (also called the frontal plane). The z-axis is through the thickness of the eyelid, along the depth of the sagittal plane (also called the longitudinal plane).

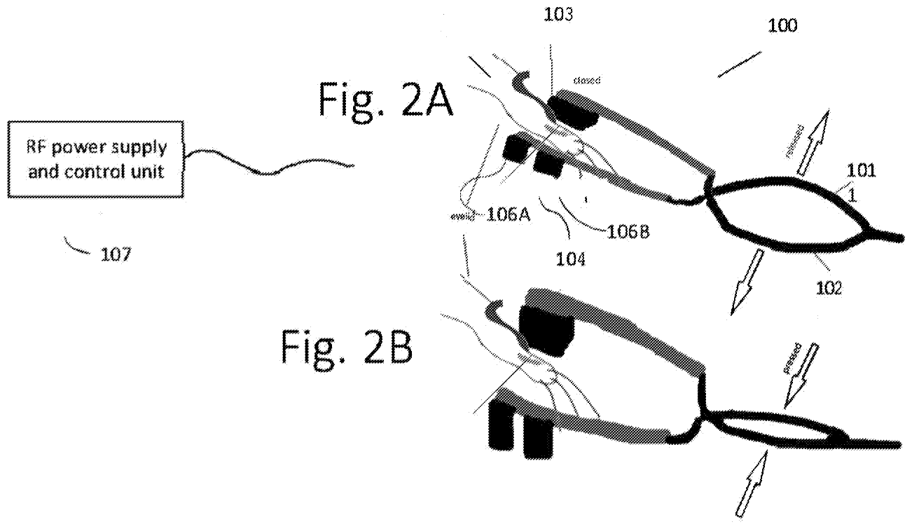

Reference is now made to FIGS. 2A-B, depicting an illustration of an eyelid treatment device 100 employing RF treatment, according to some exemplary embodiments of the invention. The device 100 may be in the form of a tweezer-like clamp. Alternatively, the device may be a tweezer with no clamping mechanism (not shown) for manual use by a user. A proximal part of the device 100, including two handles 101 and 102 held by a user, controls the opening and closing mechanism of the distal part of the device 100. The distal part includes a posterior component 103 and an anterior component 104. The posterior component 103 is inserted between the eyelid 105 and the ocular surface. The anterior component 104 is placed on the external side of the eyelid 105. The anterior component 104 includes one or more electrodes, and in this non-limiting example two electrodes 106A, 106B. Each electrode 106A, 106B is positioned over a respective underlying zone of the external side of the eyelid 105. The posterior component 103 is electrically neutral in this example and has no electrodes. In some embodiments, the posterior component 103, as will be described further below, includes one or more electrodes.

When pressing together the two handles 101, 102 of the distal part of the device 100, the posterior component 103 and anterior component 104 move apart (open state), as shown in FIG. 2B; in this open state, the device 100 can be inserted and removed from the eyelid 105. When releasing the two handles 101, 102, the posterior component 103 and the anterior component 104 may press against each other (closed state), and against the eyelid 105 when it is located in between the posterior and anterior components, as shown in FIG. 2A, thereby securing the electrodes 106A, 106B at the respective underlying zones over the eyelid 105 during treatment. After treatment, the device 100 is opened by pressing the two handles 101, 102, and can be removed from the eyelid.

During treatment, an RF field is provided by an RF power supply 107. The RF power supply 107 applies an RF signal across the electrodes 106A, 106B. In some embodiments, a control unit can be provided to control parameters of the treatment. In one non-limiting example, as shown in the Figures, the control unit is included in the RF power supply 107. In some embodiments, the control unit is configured for monitoring a temperature sensor (not shown), provided on the device 100, which provides temperature feedback to the control unit which then enables or automatically adjusts the RF signal to maintain a desired temperature during treatment. The RF power supply and/or control unit may be part of the device 100, external to the device 100, or any combination thereof.

In some embodiments of the eyelid treatment device 100, in addition to or instead of the employing RF, is configured to have at least one of the posterior component 103 and anterior component 104 as a heating element. The heating element in conjunction with the compression of the Meibomian glands of the eyelid may provide the dry eye treatment.

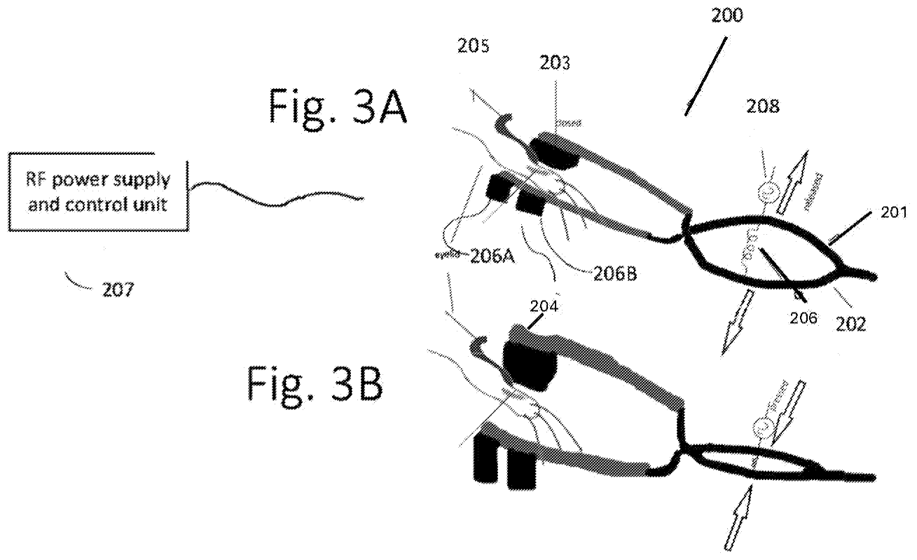

Reference is now made to FIGS. 3A-B, depicting an electromagnetically controlled RF eyelid treatment device 200, according to some non-limiting embodiments of the invention. It is noted that elements similar to the elements described in FIGS. 2A-2B have similar numbers with difference of a multiplication of 100, for example the number 201 denotes a handle similar to the handle 101. An electric circuit 208 compresses or decompresses a spring 206 connected to the handles 201, 202. When the electric circuit is turned OFF, the spring 206 is decompressed and the handles 201, 202 of the proximal part are released, allowing the posterior component 203 and anterior component 204 of the distal part to move closer to each other (in closed state). When turned ON, the electric circuit 208 activates an electromagnet that compresses the spring 206, thus bringing the handles 201, 202 together, and the posterior component 203 and front component 204 move apart (in open state). It is appreciated that the above should not be limiting and the mechanism can act in the opposite way, i.e. activating the electromagnet (the circuit is ON) compresses the spring and moves the anterior and posterior components apart and deactivating the electromagnet (the circuit is OFF) decompresses the spring and moves the anterior and posterior components towards each other.

Before placing the device 200 around the eyelid, the electric circuit 208 is turned ON. The device 200 is now in the open state, as shown in FIG. 3B. The device 200 is placed on the eyelid 205, and then the electric circuit 207 is turned OFF. The device 200 is now in the closed state as shown in FIG. 3A, and the posterior component 203 and the anterior component 204 move towards each other and press against the eyelid 205, thereby securing the electrodes 206A, 206B over the eyelid 205. At the end of a treatment, the electric circuit 207 is turned ON again to release the device 200 and enable its removal from the eyelid 205. The device 200 may then be turned OFF and stored.

As described above, during treatment, an RF field is provided by an RF power supply 207. The RF power supply 207 applies an RF signal across the electrodes 206A, 206B. A standalone or integral control unit can also be provided to control the treatment parameters such as monitoring and controlling the temperature as described above.

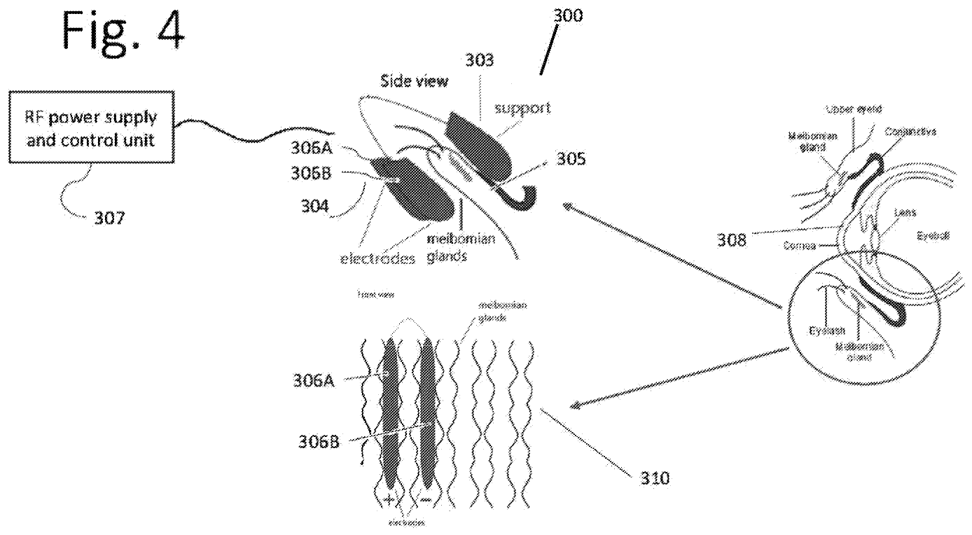

Reference is now made to FIG. 4, depicting a bipolar RF eyelid treatment device 300, according to some non-limiting embodiments of the invention. Again, it is noted that elements similar to the elements described in FIGS. 2A-2B have similar numbers with 200 differences, for example the number 306A denotes an electrode similar to the electrode 106A. The device 300 includes a posterior component 303 inserted between the eyelid 305 and the ocular surface 308, and an anterior component 304 positioned on the external side of the eyelid 305. The posterior component 303 is electrically inactive/neutral (serving to secure the device on the eyelid 305). The anterior component 304 includes two electrodes, 306A and 306B that have opposite polarities at each given time, positioned over the Meibomian glands of the eyelid 305. An RF power supply 307 applies an RF signal between the electrodes 306A and 306B. As shown in the diagram 310, an RF field propagates in the region of the eyelid 305 between the electrodes 306A and 306B, through the Meibomian glands, along the transverse axis of the eyelid 305.

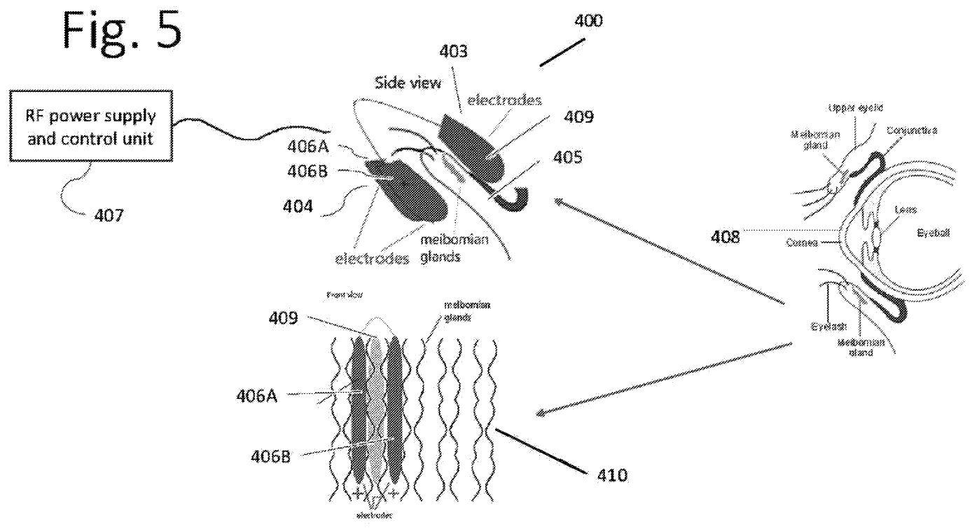

Reference is now made to FIG. 5, depicting a tripolar sagittal RF eyelid treatment device 400, according to some non-limiting embodiments of the invention. As described in the above example, the device 400 includes a posterior component 403 inserted between the eyelid 405 and the ocular surface/eyeball 408, and an anterior component 404 on the external side of the eyelid 405. The posterior component 403 includes a posterior electrode 409, having a first polarity, e.g. negative. The anterior component 404 includes two anterior electrodes 406A, 406B, having same second polarity, e.g. positive, and positioned over the Meibomian glands. An RF power supply 407 supplies an RF signal. The RF signal is applied between the electrode 409 and alternately to each of the electrodes 406A, 406B. As shown in the diagram 410, an RF field propagates between the electrode 409 and alternately to each of the electrodes 406A, 406B, through the Meibomian glands, along the sagittal axis of the eyelid 405.

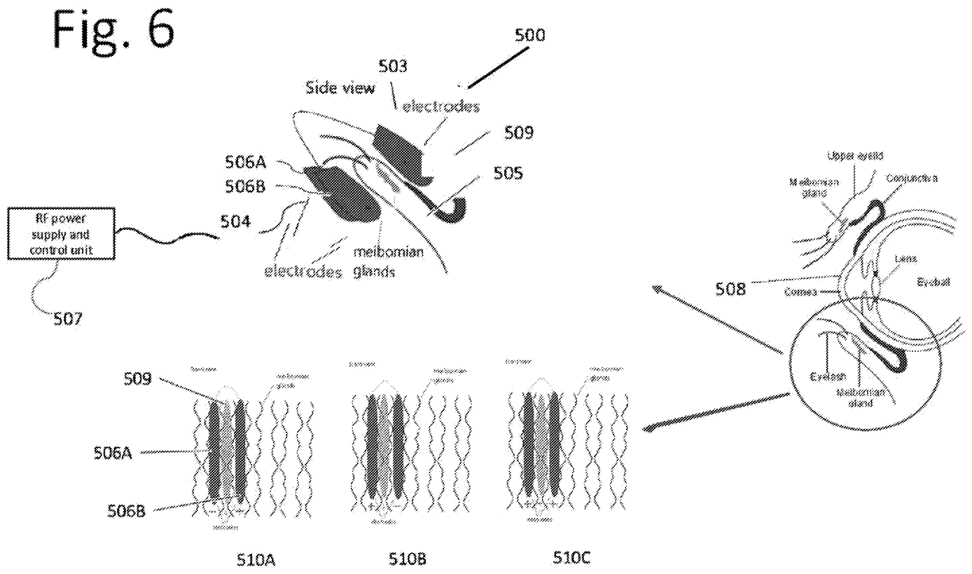

Reference is now made to FIG. 6, depicting a tripolar transverse-sagittal RF eyelid treatment device 500, according to some non-limiting embodiments of the invention. A posterior electrode 509 and two anterior electrodes 506A, 506B cyclically switch roles as the first polarity (e.g., an anode) while the other two electrodes have a second opposite polarity (e.g., are cathodes). Accordingly, the RF field cycles between the eyelid width (i.e., the transverse axis, as shown in diagrams 510A and 510B) and the eyelid thickness (i.e., the sagittal axis, as shown in diagram 510C), while at each given time two RF fields are active. In addition, it is appreciated that the three electrodes can act, even momentarily and during a treatment protocol, in a bipolar mode, i.e. with a single active RF field, where one of the electrodes has a first polarity, a second electrode has a second polarity and the third electrode is kept neutral.

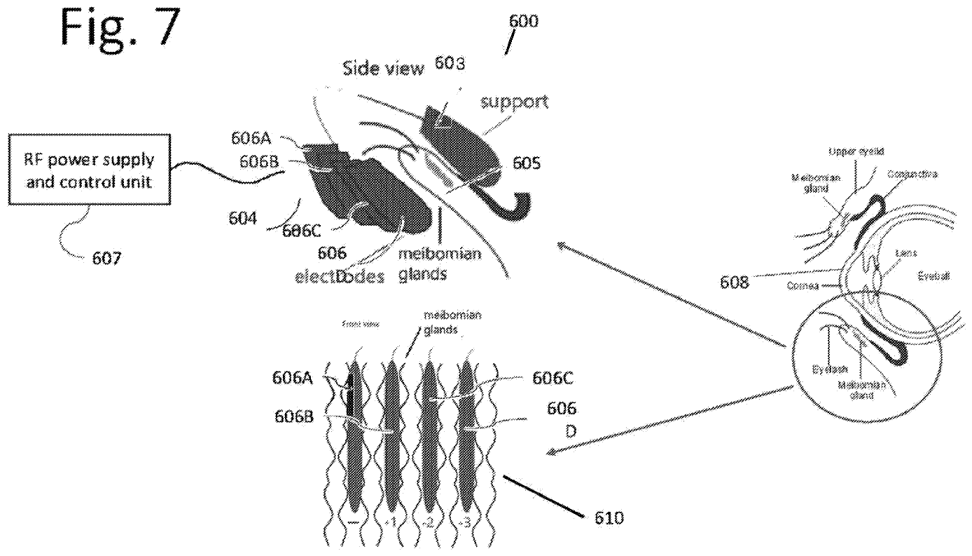

Reference is now made to FIG. 7, depicting a dynamically length-changing field bipolar RF eyelid treatment device 600, according to some non-limiting embodiments of the invention. The anterior component 604 includes an array of more than two electrodes, for example four anterior electrodes 606A-D. One of the electrodes 606A has a fixed first polarity, e.g. serves as an anode. Of the other electrodes 606B-D, one has a second opposite polarity, e.g. serves as a cathode, while the rest are electrically neutral. As shown in this non-limiting example, the cathode sequentially switches from one electrode to another electrode among the electrodes 606B-D. The RF field is always along the eyelid width 605 (i.e., the transverse axis) but the length of the RF field changes according to the cathode position with respect to the fixed anode. The diagram 610 shows four electrodes 606A-D: one electrode 606A has a first polarity (is an anode), and the other three 606B-D each sequentially has a second opposite polarity, e.g. serves as a cathode.

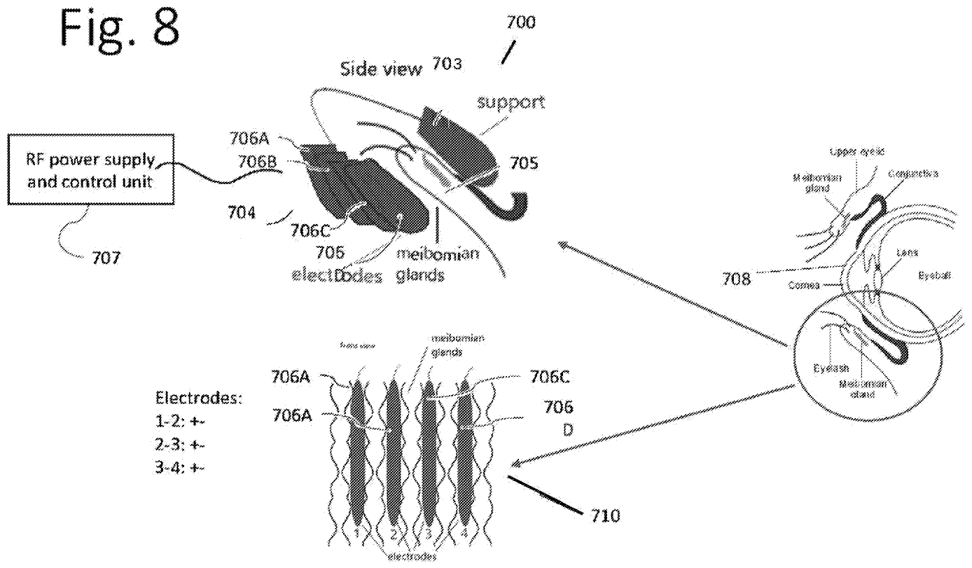

Reference is now made to FIG. 8, depicting a dynamically position-changing field bipolar RF eyelid treatment device 700, according to some embodiments of the invention. The anterior component 704 includes an array of more than two electrodes, for example four anterior electrodes 706A-D. The posterior component 703 includes an electrically neutral support. At each given time, each pair of adjacent anterior electrodes 706A-D serves as an anode/cathode pair. The RF field is along the eyelid width 705 (i.e., the transverse axis), with a sliding position. It is appreciated that the RF field can be sliding in one direction (e.g. to the right in the figure, 1-2, 2-3, 3-4) or a second direction (e.g. to the left in the figure, 4-3, 3-2, 2-1). In addition, more than one sliding cycle can be performed, e.g. starting to the right and then to the left, or a plurality of cycles to the right/left separated with a jump, e.g. 1-2, 2-3, 3-4, 1-2, 2-3, 3-4.

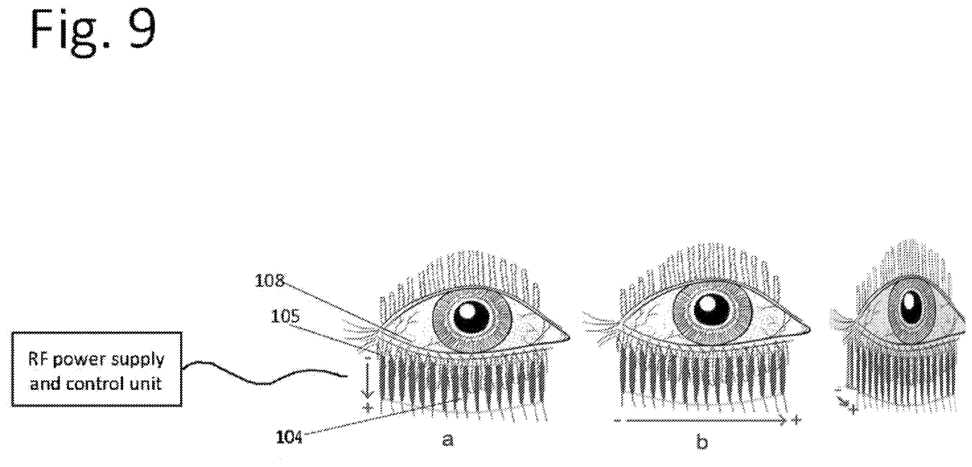

Reference is now made to FIG. 9, depicting RF directions/propagations discussed in this disclosure. Panel (a) shows RF direction along the eyelid height (i.e., the coronal axis); panel (b) shows RF direction along the eyelid width (i.e., the transverse axis); and panel (c) shows RF direction along the eyelid thickness (i.e., the sagittal axis).

1. A device for treating an eyelid with radio-frequency (RF) radiation, comprising:
a posterior component configured to be positioned over the internal side of the eyelid and an anterior component configured to be positioned on the external side of the eyelid;
at least two electrodes, comprising any combination of one or more posterior electrodes mounted on said posterior component to be positioned on said internal side of the eyelid and/or one or more anterior electrodes mounted on said anterior component to be positioned on said external side of the eyelid, each said electrode configured to be positioned over an underlying zone of the eyelid;
the device being configured for the at least two electrodes to provide an RF field to the eyelid along one or more of a coronal, transverse, and sagittal axis of the eyelid when an RF signal is applied between said at least two electrodes.

2. The device of claim 1, further comprising a clamping mechanism configured to move the posterior and anterior components away from each other, thereby enabling insertion and removal of the device from the eyelid, and to compress the posterior and anterior components against each other, thereby securing said at least two electrodes to the respective underlying zones.

3. The device of claim 2, wherein the clamping mechanism is manually operated.

4. The device of claim 2, wherein the clamping mechanism is electrically operated.

5. The device of claim 2, wherein the clamping mechanism is magnetically operated.

6. The device of claim 2, wherein when the clamping mechanism is activated, the posterior and anterior components are moved away from each other, and when the clamping mechanism is deactivated, the posterior and anterior components are compressed against each other.

7. The device of claim 1, wherein the at least two electrodes comprise two anterior electrodes positioned on said external side of the eyelid, the device thereby being configured to provide said RF field along the transverse axis of the eyelid.

8. The device of claim 7, wherein the at least two electrodes comprise at least three anterior electrodes positioned on said external side of the eyelid along the transverse axis with a fixed distance between each two adjacent anterior electrodes, the device thereby being configured to provide one of the following effects:
a) a sliding RF field along the transverse axis of the eyelid by sequentially activating two adjacent anterior electrodes of the at least three anterior electrodes;
b) a length-varying RF field along the transverse axis of the eyelid by activating at each given time the most right electrode or the most left electrode of the anterior electrodes at a first polarity while sequentially activating the rest of the anterior electrodes at an opposite second polarity.

9. The device of claim 1, wherein said at least two electrodes comprise at least one anterior electrode positioned on said external side of the eyelid and at least one posterior electrode positioned on said internal side of the eyelid; the device thereby being configured to provide said RF field along the sagittal axis of the eyelid between the at least one anterior and at least one posterior electrodes.

10. The device of claim 9, wherein said at least two electrodes comprise two anterior electrodes, the device thereby being configured to alternate said RF field along the sagittal axis of the eyelid between each of said two anterior electrodes and said at least one posterior electrode.

11. The device of claim 1, being at least one of; disposable, or partially disposable.

12. The device of claim 1, comprising a disposable sleeve configured to cover at least said at least two electrodes.

13. The device of claim 1, wherein the posterior component is electrically inactive, and the anterior component comprises two electrodes that have opposite polarities to each other, such that when an RF signal is applied between the two electrodes, an RF field propagates between the two electrodes.

14. The device of claim 1, wherein:
the posterior component comprises a posterior electrode with a first polarity, and the anterior component comprises two anterior electrodes both comprising a second polarity; and
the posterior electrode and the two anterior electrodes are further configured to apply the RF signal between the posterior electrode alternately to each of the two anterior electrodes.

15. The device of claim 14, wherein the posterior electrode is further configured to cyclically switch from a first polarity to a second polarity and the two anterior electrodes are further configured to cyclically switch from a second polarity to a first polarity in sync with the switch of the posterior electrode.

16. The device of claim 1, wherein the posterior component is electrically inactive, and the anterior component comprises one anterior electrode fixed in a first polarity, and at least two additional anterior electrodes and the at least two additional anterior electrodes are configured to switch between a second plurality and an electrically inactive status.

17. A device for treating an eyelid, comprising:
a posterior component configured to be positioned over the internal side of the eyelid and an anterior component configured to be positioned on the external side of the eyelid;
said posterior component to be positioned on said internal side of the eyelid said anterior component to be positioned on said external side of the eyelid;
at least one of the posterior component or the anterior component configured to be a heating element to provide heating of the Meibomian glands of the eyelid.

18. The device of 17, wherein the device is further configured to allow a user to compress an eyelid between the posterior component and the anterior component.

19. The device of 17, further comprising at least two electrodes, comprising any combination of one or more posterior electrodes mounted on said posterior component and/or one or more anterior electrodes mounted on said anterior component, each said electrode configured to be positioned over an underlying zone of the eyelid.
